(19) United States
(12) Patent Application Publication
(10) Pub. No.: US 2009/0270497 A1
(43) Pub. Date: Oct. 29, 2009

(54) Treatment of Non-Localized Inflammation with pan-HDAC Inhibitors

(75) Inventor: Joseph J. Buggy, Mountain View, CA (US)

Correspondence Address: WILSON, SONSINI, GOODRICH & ROSATI, 650 PAGE MILL ROAD, PALO ALTO, CA, 94304-1050, US

(73) Assignee: PHARMACYCLICS, INC., Sunnyvale, CA (US)

(21) Appl. No.: 12/429,996

(22) Filed: Apr. 24, 2009

Related U.S. Application Data
(60) Provisional application No. 61/047,565, filed on Apr. 24, 2008.

(51) Int. Cl. A61K 31/343; A61P 31/04

(52) U.S. Cl. 514/469

(57) ABSTRACT

Described herein are compositions and methods for treating a subject suffering from a non-localized inflammatory condition (or any symptoms associated with such inflammation), including systemic inflammation, and inflammatory conditions affecting the large portions of or the whole body, or sepsis by administering to the subject a pharmaceutical composition comprising a therapeutically effective amount of a compound that is a pan-HDAC inhibitor. Also described herein are methods for decreasing iNOS and cytokine expression by administering to the subject a pharmaceutical composition comprising a therapeutically effective amount of a compound that is a pan-HDAC inhibitor.

### RELATED APPLICATIONS

This application claims benefit of U.S. Provisional Application No. 61/047,565 entitled “TREATMENT OF NON-LOCALIZED INFLAMMATION WITH PAN-HDAC INHIBITORS” filed Apr. 24, 2008, which is herein incorporated by reference.

### FIELD OF THE INVENTION

Described herein are pharmaceutical compositions comprising pan-HDAC inhibitors (pan-HDACi) and methods of use for inhibiting the activity of a plurality of histone deacetylases as a treatment for non-localized inflammatory conditions, including systemic inflammation, and inflammatory conditions affecting the large portions of or the whole body. Further described herein are methods for using a pan-HDACi as a treatment for sepsis. Further described herein are methods for decreasing iNOS expression with a pan-HDACi.

### BACKGROUND

Histones are proteins that organize and modulate the structure of chromatin in nucleosomes. Histone deacetylases (HDACs) were originally identified as proteins that catalyze the removal of acetyl groups from histones. HDAC-mediated deacetylation of chromatin-bound histones and other acetylated protein substrates (e.g., tubulin) participates in cell signaling. To date eleven isoforms of HDAC have been described (HDACs 1-11).

### SUMMARY OF THE INVENTION

Described herein are methods for treating non-localized inflammatory conditions (or any symptoms associated with such inflammation), including systemic inflammation, and inflammatory conditions affecting the large portions of or the whole body, in a subject in need thereof. In some embodiments, the method comprises administering to the subject a pharmaceutical composition comprising a therapeutically effective amount of a pan-HDAC inhibitor wherein the pan-HDAC inhibitor has Formula (I):

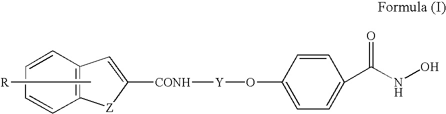

wherein Z is S, O, or NH; Y is an alkylene optionally substituted with cycloalkyl, optionally substituted phenyl, alkylthio, alkylsulfinyl, alkylsulfonyl, optionally substituted phenylalkylthio, optionally substituted phenylalkylsulfonyl, hydroxyl, or optionally substituted phenoxy; R is one or two optional substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenyl, optionally substituted phenoxy, optionally substituted phenylalkyloxy, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)ₙR^{a} (where n is 0, 1 or 2 and R^{a} is hydroxyalkyl or optionally substituted phenyl), -alkylene-NR^{e}-alkyleneCONR^{c}R^{d} (where R^{c} is hydroxyl and R^{d} and R^{e} are independently hydrogen or alkyl), or carboxyalkylaminoalkyl.

In some embodiments, Z is O, NH, or S; and Y is —CH₂CH₂—.

In some embodiments, Z is O (i.e., a benzofuranyl group). In some embodiments, the benzofuranyl group is monosubstituted.

In some embodiments, Z is N (i.e., an indolyl group). In some embodiments, the indolyl group is monosubstituted.

In some embodiments, Z is S (i.e., a benzothiofuranyl group). In some embodiments, the benzothiofuranyl group is monosubstituted.

In some embodiments, substituent on the benzofuranyl group, the indolyl group, or the benzothiofuranyl group is N,N-dimethylaminomethyl, N,N-diethylaminomethyl, 2-fluorophenoxymethyl, 3-fluorphwenoxymethyl, 4-fluorophenoxymethyl, hydroxyl-4-yloxymethyl, 2,4,6-trifluorophenoxy-methyl, 2-oxopyridin-1-ylmethyl, 2,2,2-trifluorethoxy-methyl, 4-imidazol-1-ylphenoxy-methyl, 4-[1.2.4]-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenyl)ethyl, N-methyl-N-benzylaminomethyl, N-methyl-N-2-phenylethylaminomethyl, 3-hydroxypropyl-thiomethyl, 3-hydroxypropylsulfinylmethyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylaminomethyl.

In some embodiments, substituent on the benzofuranyl group, the indolyl group, or the benzothiofuranyl group is N,N-dimethylaminomethyl.

In some embodiments, the expression of iNOS decreases or is down-regulated following administration of the pharmaceutical composition. In some embodiments, the concentration of nitric oxide in the blood of the subject decreases following administration of the pharmaceutical composition. In some embodiments, after administration of the pharmaceutical composition, the blood pressure of the subject increases. In some embodiments, following administration of the pharmaceutical composition the expression of one or more cytokines has decreased or is down-regulated. In some embodiments, the one or more cytokines is selected from the group consisting of: IL-1, IL-6, TNF-α, MCP-1, any isoforms thereof, and any combinations thereof.

In some embodiments, the pharmaceutical composition is administered in combination with one or more additional therapeutic agents. In some embodiments, the one or more additional therapeutic agents is selected from the group consisting of: immunosuppressants, antibiotics, glucocorticoids, non-steroidal anti-inflammatory drugs, Cox-2-specific inhibitors, disease modifying antirheutetic drugs, TNF-α binding proteins, beta-agonists, and any combinations thereof.

In some embodiments, the subject is a human.

Described herein are methods for treating sepsis (or any of the symptoms associated with sepsis) in a subject in need thereof.

In one embodiment, described herein is a method for treating sepsis in a subject comprising administering to the subject a pharmaceutical composition comprising a therapeutically effective amount of a pan-HDAC inhibitor to the subject in need thereof.

In some embodiments, the pan-HDAC inhibitor is a short-chain fatty acid pan-HDAC inhibitor, hydroxamic acid pan-HDAC inhibitor, epoxyketone-containing cyclic tetrapeptide pan-HDAC inhibitor, benzamide pan-HDAC inhibitor, cyclic-hydroxamic-acid-containing peptide (CHAP) pan-HDAC inhibitor, benzamide pan-HDAC inhibitor, depudecin, organosulfur pan-HDAC inhibitor, or an aroyl-pyrrolylhydroxy-amide (APHA) pan-HDAC inhibitor.

In some embodiments, the pan-HDAC inhibitor is butyrate, 4-phenylbutyrate, valproic acid, suberoylanilide hydroxamic acid (SAHA), biaryl hydroxamate A-161906, bicyclic aryl-N-hydroxycarboxamides, CG-1521, PXD-101, sulfonamide hydroxamic acid, LAQ-824, oxamflatin, scriptaid, m-carboxy cinnamic acid bishydroxamic acid, trapoxin-hydroxamic acid analogue, trichostatin A, trichostatin C, m-carboxycinnamic acid bis-hydroxamideoxamflatin (CBHA), azelaic bishydroxamic acid (ABHA), Scriptaid, Sirtinol, pyroxamide, trapoxins, apidicin, depsipeptide, HC-toxin, chlamydocin, diheteropeptin, WF-3161, Cyl-1 and Cyl-2, FR901228, apicidin, cyclic-hydroxamic-acid-containing peptide (CHAP), MS-275 (MS-27-275), CI-994, depudecin, PXD101, an aroyl-pyrrolylhydroxy-amide (APHA), LBH-589, MGCD-0103, JNJ-26481585, R306465 (J&J), or sodium butyrate.

In some embodiments, the pan-HDAC inhibitor has the structure of Formula (I):

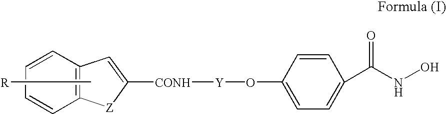

wherein Z is S, O, or NH; Y is an alkylene optionally substituted with cycloalkyl, optionally substituted phenyl, alkylthio, alkylsulfinyl, alkylsulfonyl, optionally substituted phenylalkylthio, optionally substituted phenylalkylsulfonyl, hydroxyl, or optionally substituted phenoxy; R is one or two optional substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenyl, optionally substituted phenoxy, optionally substituted phenylalkyloxy, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)ₙR^{a} (where n is 0, 1 or 2 and R^{a} is hydroxyalkyl or optionally substituted phenyl), -alkylene-NR^{e}-alkyleneCONR^{c}R^{d} (where R^{c} is hydroxyl and R^{d} and R^{e} are independently hydrogen or alkyl), or carboxyalkylaminoalkyl.

In some embodiments, Z is O, NH, or S; and Y is —CH₂CH₂—.

In some embodiments, Z is O (i.e., a benzofuranyl group). In some embodiments, the benzofuranyl group is monosubstituted with R.

In some embodiments, Z is N (i.e., an indolyl group). In some embodiments, the indolyl group is monosubstituted with R.

In some embodiments, Z is S (i.e., a benzothiofuranyl group). In some embodiments, the benzothiofuranyl group is monosubstituted with R.

In some embodiments, substituent on the benzofuranyl group, the indolyl group, or the benzothiofuranyl group is N,N-dimethylaminomethyl, N,N-diethylaminomethyl, 2-fluorophenoxymethyl, 3-fluorphwenoxymethyl, 4-fluorophenoxymethyl, hydroxyl-4-yloxymethyl, 2,4,6-trifluorophenoxy-methyl, 2-oxopyridin-1-ylmethyl, 2,2,2-trifluorethoxy-methyl, 4-imidazol-1-ylphenoxy-methyl, 4-[1.2.4]-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenyl)ethyl, N-methyl-N-benzylaminomethyl, N-methyl-N-2-phenylethylaminomethyl, 3-hydroxypropyl-thiomethyl, 3-hydroxypropylsulfinylmethyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylaminomethyl.

In some embodiments, substituent on the benzofuranyl group, the indolyl group, or the benzothiofuranyl group is N,N-dimethylaminomethyl.

In some embodiments, the pan-HDAC inhibitor is Compound 1:

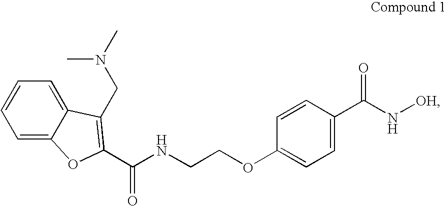

or a pharmaceutically acceptable salt thereof.

In some embodiments, the pan-HDAC inhibitor is the HCl salt of Compound 1.

A pan-HDAC inhibitor for treating sepsis in a human. In one aspect, the pan-HDAC inhibitor is described herein. In one aspect, the pan-HDAC inhibitor is a compound with the structure of Formula (I), or a pharmaceutically acceptable salt thereof. In one aspect, the pan-HDAC inhibitor is Compound 1, or a pharmaceutically acceptable salt thereof. In one aspect, the pan-HDAC inhibitor is the HCl salt of Compound 1.

Also described is the use of a pan-HDAC inhibitor in the manufacture of a medicament for treating sepsis in a human. In some embodiments, the pan-HDAC inhibitor is a short-chain fatty acid pan-HDAC inhibitor, hydroxamic acid pan-HDAC inhibitor, epoxyketone-containing cyclic tetrapeptide pan-HDAC inhibitor, benzamide pan-HDAC inhibitor, cyclic-hydroxamic-acid-containing peptide (CHAP) pan-HDAC inhibitor, benzamide pan-HDAC inhibitor, depudecin, organosulfur pan-HDAC inhibitor, or an aroyl-pyrrolylhydroxy-amide (APHA) pan-HDAC inhibitor.

In some embodiments, the pan-HDAC inhibitor is butyrate, 4-phenylbutyrate, valproic acid, suberoylanilide hydroxamic acid (SAHA), biaryl hydroxamate A-161906, bicyclic aryl-N-hydroxycarboxamides, CG-1521, PXD-101, sulfonamide hydroxamic acid, LAQ-824, oxamflatin, scriptaid, m-carboxy cinnamic acid bishydroxamic acid, trapoxin-hydroxamic acid analogue, trichostatin A, trichostatin C, m-carboxycinnamic acid bis-hydroxamideoxamflatin (CBHA), azelaic bishydroxamic acid (ABHA), Scriptaid, Sirtinol, pyroxamide, trapoxins, apidicin, depsipeptide, HC-toxin, chlamydocin, diheteropeptin, WF-3161, Cyl-1 and Cyl-2, FR901228, apicidin, cyclic-hydroxamic-acid-containing peptide (CHAP), MS-275 (MS-27-275), CI-994, depudecin, PXD101, an aroyl-pyrrolylhydroxy-amide (APHA), LBH-589, MGCD-0103, JNJ-26481585, R306465 (J&J), or sodium butyrate.

In some embodiments, the pan-HDAC inhibitor is a compound with the structure of Formula (I), or a pharmaceutically acceptable salt thereof.

In some embodiments, the pan-HDAC inhibitor is Compound 1, or a pharmaceutically acceptable salt thereof. In some embodiments, the pan-HDAC inhibitor is the HCl salt of Compound 1.

In some embodiments, the expression of iNOS decreases or is down-regulated following administration of the pharmaceutical composition. In some embodiments, the concentration of nitric oxide in the blood of the subject decreases following administration of the pharmaceutical composition. In some embodiments, after administration of the pharmaceutical composition, the blood pressure of the subject increases. In some embodiments, following administration of the pharmaceutical composition the expression of one or more cytokines has decreased or is down-regulated. In some embodiments, the one or more cytokines is selected from the group consisting of: IL-1, IL-6, TNF-α, MCP-1, any isoforms thereof, and any combinations thereof.

In some embodiments, the pharmaceutical composition is administered in combination with one or more additional therapeutic agents. In some embodiments, the one or more additional therapeutic agents is selected from the group consisting of: immunosuppressants, antibiotics, glucocorticoids, non-steroidal anti-inflammatory drugs, Cox-2-specific inhibitors, disease modifying antirheutetic drugs, TNF-α binding proteins, beta-agonists, and any combinations thereof.

In some embodiments, the subject is a human.

In one aspect, the pharmaceutical composition is formulated for intravenous administration, subcutaneous injection, oral administration, or inhalation.

In one aspect, the pharmaceutical composition is formulated for intravenous administration or subcutaneous injection. In one aspect, the pharmaceutical composition is formulated for oral administration.

In any of the aforementioned aspects are further embodiments comprising multiple administrations of the effective amount of the pan-HDAC inhibitor, including further embodiments in which (i) the pan-HDAC inhibitor is administered continuously or intermittently: as in a single dose; (ii) the time between multiple administrations is every 6 hours; (iii) the pan-HDAC inhibitor is administered to the subject every 8 hours. In some embodiments, the method comprises a drug holiday, wherein the administration of the pan-HDAC inhibitor is temporarily suspended or the dose of the pan-HDAC inhibitor being administered is temporarily reduced; at the end of the drug holiday, dosing of the pan-HDAC inhibitor is resumed. In one embodiment, the length of the drug holiday varies from 2 days to 1 year.

In one aspect, the pharmaceutical composition is a tablet, a pill, a capsule, a liquid, an inhalant, a solution, a gel, a colloid, a dispersion, a suspension, or an emulsion. In one aspect, the pharmaceutical composition is a tablet, a pill, a capsule, a liquid, a solution, a dispersion, a suspension, or an emulsion.

In any of the aforementioned aspects are further embodiments in which: (a) the effective amount of the pan-HDAC inhibitor is systemically administered to the subject; and/or (b) the effective amount of the pan-HDAC inhibitor is administered orally to the subject; and/or (c) the effective amount of the pan-HDAC inhibitor is intravenously administered to the subject; and/or (d) the effective amount of the pan-HDAC inhibitor is administered by injection to the mammal.

In any of the aforementioned aspects are further embodiments comprising single administrations of the effective amount of the pan-HDAC inhibitor, including further embodiments in which (i) the pan-HDAC inhibitor is administered once; (ii) the pan-HDAC inhibitor is administered to the subject multiple times over the span of one day; (iii) continually; or (iv) continuously.

In any of the aforementioned embodiments involving the treatment with a pan-HDAC inhibitor are further embodiments comprising administering at least one additional agent in addition to the administration of the pan-HDAC inhibitor. Each agent is administered in any order, including simultaneously.

In some embodiments, the pharmaceutical composition comprising a pan-HDAC inhibitor is administered to a human.

In some embodiments, the pharmaceutical composition comprising a pan-HDAC inhibitor is orally administered.

In some embodiments, the pharmaceutical composition comprising a pan-HDAC inhibitor is administered intraveneously.

Other objects, features and advantages of the methods, compounds, and compositions described herein will become apparent from the following detailed description. It should be understood, however, that the detailed description and the specific examples, while indicating specific embodiments, are given by way of illustration only, since various changes and modifications within the spirit and scope of the disclosure will become apparent to those skilled in the art from this detailed description.

### DESCRIPTION OF DRAWINGS

FIG. 1 is an illustrative immunoblot and corresponding bar graph showing pan-HDACi Compound 1 expression in a series of cell lines. Compound 1 decreases RNA levels of TNFα, IL-1β, and IL-6, but not GAPDH.

FIG. 2 is an illustrative bar graph showing Compound 1 expression in a series of cell lines. Compound 1 decreases cytokine expression of TNFα, IL-1β and IL-6 proteins.

FIG. 3 is an illustrative immunoblot showing Compound 1 decreases iNOS expression. Cell lysates were analyzed by Western blot with antibodies to the inducible nitric oxide synthase (iNOS) (above) and HuR (below) as negative control.

FIG. 4 is an illustrative line graph showing Compound 1 decreases mortality from LPS. Compound 1 increases survival from endotoxemia by 70%.

### DETAILED DESCRIPTION OF THE INVENTION

### Non-Localized Inflammation and Sepsis

Sepsis is, broadly defined, a medical condition characterized by the presence of pathogens in the blood (septicemia) or tissues of a subject. For example, sepsis caused by a bacterial infection is called bacteremia; sepsis caused by a viral infection is called viremia; sepsis caused by a fungal infection is called fungemia. Sepsis is often characterized by acute inflammation of the whole body and, therefore, the patient may also present with a fever and an elevated white blood cell count (leukocytosis). It is often the responses of the patient's immune system to the infection (such as fever and inflammation) which causes the most severe and lasting damage to the patient (such as damage to the vasculature and organs). The mortality rate for patients with sepsis can be as high as 60%.

Sepsis is considered present if (a) infection is confirmed, and (b) two or more of the systemic inflammatory response syndrome (SIRS) criteria are met. Confirmation of infection can come from culture, stain, polymerase chain reaction (PCR), or it can be confirmed if the subject displays symptoms consistent with the infection. Symptoms consistent with the presence of an infectious agent include, but are not limited to, white blood cells in normally sterile fluid, evidence of a perforated viscus, abnormal chest x-ray consistent with pneumonia (with focal opacification), or petechiae, purpura, or purpura fulminans.

By way of example, the systemic inflammatory response syndrome (SIRS) criteria for adults are: a heart rate greater than 90 beats per minute (tachycardia); body temperature less than 36° C. (96.8° F.) or greater than 38° C. (100.4° F.) (hyperthermia or fever); a respiratory rate of greater than 20 breaths per minute or, if measured by blood gas, a PₐCO₂ less than 32 mm Hg (4.3 kPa) (tachypnea or hypocapnia due to hyperventilation); and a white blood cells count that is less than 4000 cells/mm³ or greater than 12000 cells/mm³ (<4×10⁹ or >12×10⁹ cells/L), or greater than 10% band forms (immature white blood cells) (leukopenia, leukocytosis, or bandemia). These criteria are modified for children.

Non-localized inflammatory conditions include, but are not limited to, sepsis, systemic inflammatory response syndrome (SIRS), cytokine storms, septic shock, rheumatic fever, and systemic lupus erythematosus (SLE).

Systemic inflammatory response syndrome (SIRS) is an inflammatory state of the whole body without a proven source of infection.

### Inflammation, Cytokines, and the Immune Response

Inflammation is a biological response to an injury or an infection. A tissue becomes inflamed when plasma and/or leukocytes move to the site of infection or injury. Swelling, a common symptom of inflammation, results from the movement of plasma and/or leukocytes to the site of the inflection or injury.

Some leukocytes act as phagocytes, ingesting bacteria, viruses, fungi, and cellular debris. Others release enzymatic granules which damage pathogenic invaders. Leukocytes also release inflammatory mediators which develop and maintain the inflammatory response.

Cytokines are signaling proteins or glycoproteins. They are secreted by certain cells of the body (for example, the immune system cells neutrophil granulocytes and macrophages) in response to an infection. Most cytokines range in size from about 8 kDa to about 30 kDa.

When an immune system cell encounters a pathogen, the cell secretes cytokines as a means to signal and activate other immune cells. Each cytokine binds to a specific cell-surface receptor. The binding of the cytokine to the receptor can result in the upregulation or downregulation of certain genes and their transcription factors. This may in turn cause the production of, among other things, more cytokines or an increase in the cell surface receptors.

By way of non-limiting example, cytokines may include interleukins IL-1, IL-6, IL-8, MCP-1 (also known as CCL2), and TNF-α. Interleukin 1 is present in the body in two isoforms: IL-1α and IL-1β. Both isoforms are produced by macrophages, monocytes and dendritic cells. It has been shown that the presence of either IL-1 isoform increases the expression of adhesion factors on endothelial cells. This, in turn, enables the transmigration of leukocytes to the site of infection. IL-1 has also been shown to reset the hypothalamus thermoregulatory center, leading to an increased body temperature.

An increase in IL-6 levels is often correlated with fever. Fever is the result of Il-6 stimulating energy mobilization in muscle and fatty tissues. IL-6 can be secreted by macrophages in response to the presence of a pathogen.

IL-8 has been shown to induce the migration of neutrophils to the site of an infection (a process called chemotaxis). Cell surface receptors on the neutrophils are able to detect chemical gradients of IL-8 (among other chemicals). The neutrophil follows the gradient to the site of infection. There the neutrophil engages in the phagocytosis of pathogens. Neutrophils are the primary cell found in pus.

TNF-α is a key mediator of septic shock. It is mainly produced by macrophages, but is also produced by lymphoid cells, mast cells, endothelial cells, cardiac myocytes, adipose tissue, fibroblasts and neuronal tissue. TNF is released in response to lipopolysaccharide (LPS), other bacterial products, and Interleukin-1. It attracts neutrophils, and participates in the chemotaxis of the neutrophils. It stimulates phagocytosis by macrophages, and the production of IL-1 oxidants and the inflammatory lipid prostaglandin E2 (PGE₂).

MCP-1 (also known as CCL2) is often found at the site of a tooth eruption or bone degradation. MCP-1 is expressed by mature osteoclasts and osteoblasts. It recruits immune cells, such as monocytes, T lymphocytes, eosinophils, and basophils to sites of tissue injury and infection. It has been associated with many inflammatory reactions to disease. Finally, it has been linked to recruitment of osteoclast precursors.

Described herein are methods for treating non-localized inflammatory conditions (or any symptoms associated with such inflammation), including systemic inflammation, and inflammatory conditions affecting the large portions of or the whole body in a subject in need thereof. In some embodiments, the method comprises administering to the subject a pharmaceutical composition comprising a therapeutically effective amount of a pan-HDAC inhibitor wherein the pan-HDAC inhibitor has Formula (I):

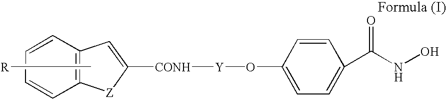

wherein Z is S, O, or NH; Y is an alkylene optionally substituted with cycloalkyl, optionally substituted phenyl, alkylthio, alkylsulfinyl, alkylsulfonyl, optionally substituted phenylalkylthio, optionally substituted phenylalkylsulfonyl, hydroxyl, or optionally substituted phenoxy; R is one or two optional substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenyl, optionally substituted phenoxy, optionally substituted phenylalkyloxy, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)ₙR^{a} (where n is 0, 1 or 2 and R^{a} is hydroxyalkyl or optionally substituted phenyl), -alkylene-NR^{e}-alkyleneCONR^{c}R^{d} (where R^{c} is hydroxyl and R^{d} and R^{e} are independently hydrogen or alkyl), or carboxyalkylaminoalkyl.

In some embodiments, Z is O, NH, or S; and Y is —CH₂CH₂—.

In some embodiments, Z is O (i.e., a benzofuranyl group), and in some embodiments, the benzofuranyl group is monosubstituted.

In some embodiments, Z is N (i.e., an indolyl group), and in some embodiments, the indolyl group is monosubstituted.

In some embodiments, Z is S (i.e., a benzothiofuranyl group), and in some embodiments, the benzothiofuranyl group is monosubstituted.

In some embodiments, substituent on the benzofuranyl group, the indolyl group, or the benzothiofuranyl group is N,N-dimethylaminomethyl, N,N-diethylaminomethyl, 2-fluorophenoxymethyl, 3-fluorphwenoxymethyl, 4-fluorophenoxymethyl, hydroxyl-4-yloxymethyl, 2,4,6-trifluorophenoxy-methyl, 2-oxopyridin-1-ylmethyl, 2,2,2-trifluorethoxy-methyl, 4-imidazol-1-ylphenoxy-methyl, 4-[1.2.4]-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenyl)ethyl, N-methyl-N-benzylaminomethyl, N-methyl-N-2-phenylethylaminomethyl, 3-hydroxypropyl-thiomethyl, 3-hydroxypropylsulfinylmethyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylaminomethyl.

In some embodiments, the expression of iNOS decreases or is down-regulated following administration of the pharmaceutical composition. In some embodiments, the concentration of nitric oxide in the blood of the subject decreases following administration of the pharmaceutical composition. In some embodiments, after administration of the pharmaceutical composition, the blood pressure of the subject increases. In some embodiments, following administration of the pharmaceutical composition the expression of one or more cytokines has decreased or is down-regulated. In some embodiments, the one or more cytokines is selected from the group consisting of: IL-1, IL-6, TNF-α, MCP-1, any isoforms thereof, and any combinations thereof. In some embodiments, the pharmaceutical composition is administered in combination with one or more additional therapeutic agents. In some embodiments, the one or more additional therapeutic agents is selected from the group consisting of: immunosuppressants, antibiotics, glucocorticoids, non-steroidal anti-inflammatory drugs, Cox-2-specific inhibitors, disease modifying antirheutetic drugs, TNF-α binding proteins, beta-agonists, and any combinations thereof. In some embodiments, the subject is a human patient.

Described herein are methods for treating sepsis (or any of the symptoms associated with sepsis) in a subject in need thereof. In some embodiments, the method comprises administering to the subject a pharmaceutical composition comprising a therapeutically effective amount of a pan-HDAC inhibitor wherein the pan-HDAC inhibitor has Formula (I):

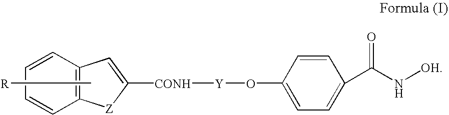

In some embodiments, the method comprises administering to the subject a pharmaceutical composition comprising a therapeutically effective amount of Compound 1.

In some embodiments, the method comprises administering to the subject a pharmaceutical composition comprising a therapeutically effective amount of the HCl salt of Compound 1.

### CERTAIN DEFINITIONS

Unless otherwise stated, the following terms used in the specification and claims are defined for the purposes of this application and have the following meanings:

As used herein, “derivative” means a compound that is produced from another compound or similar structure by the replacement or substitution of an atom, molecule, or group by another atom, molecule, or group. By way of non-limiting example, if a pan-HDAC inhibitor contains an oxidizable nitrogen atom, the nitrogen atom may be converted to an N-oxide by known methods to produce an N-oxide derivative. By way of further non-limiting example, if a pan-HDAC inhibitor contains a hydroxy group, a carboxy group, a thiol group, or any group containing one or more nitrogen atoms these groups may be protected with suitable protecting groups to produce a protected derivative. A list of suitable protective groups is found in T. W. Greene, Protective Groups in Organic Synthesis, John Wiley & Sons, Inc. 1981.

As used herein, “effective amount,” or “therapeutically effective amount” means an amount of an agent which confers a pharmacological effect or therapeutic effect on a subject without undue adverse side effects. It is understood that the effective amount or the therapeutically effective amount will vary from subject to subject, based on the subject's age, weight, and general condition, the condition being treated, the severity of the condition being treated, and the judgment of the prescribing physician.

As used herein, “histone deacetylase” and “HDAC” refer to any one of a family of enzymes that remove acetyl groups from the ε-amino groups of lysine residues at the N-terminus of a histone. Unless otherwise indicated, the term “histone” means any histone protein, including H1, H2A, H2B, H3, H4, and H5, from any species. Human HDAC proteins or gene products, include, but are not limited to, HDAC-1, HDAC-2, HDAC-3, HDAC4, HDAC-5, HDAC-6, HDAC-7, HDAC-8, HDAC-9, HDAC-10, and HDAC-11. In some embodiments, the HDAC is also derived from a protozoal or fungal source.

As used herein, “histone deacetylase inhibitor,” “inhibitor of histone deacetylase,” “HDAC inhibitor,” “inhibitor of HDAC,” and HDACi are used interchangeably to identify a compound, which is capable of interacting with a HDAC and inhibiting its activity, more particularly its enzymatic activity. Inhibiting HDAC enzymatic activity means reducing the ability of a HDAC to remove an acetyl group from a histone. In some embodiments, such inhibition is specific, i.e. the HDAC inhibitor reduces the ability of a HDAC to remove an acetyl group from a histone at a concentration that is lower than the concentration of the inhibitor that is required to produce some other, unrelated biological effect.

As used herein, “pan-HDAC inhibitor” and “pan-HDACi” means (a) a chemical or biological agent that inhibits all eleven HDAC isoforms of the class I and class II enzymes, or (b) a chemical or biological agent that significantly inhibits more that one isoform of class I or class II HDACs with a Ki of less than 1 μM.

As used herein, “subject” means a human or animal in need of treatment for a condition which may be treatable by one of the pan-HDAC inhibitors described herein.

As used herein, “treat,” “treating,” or “treatment” refers to, but is not limited to, inhibiting the progression of a disorder or disease, for example arresting the development of the disease or disorder. By way of non-limiting example, treatment of non-localized inflammatory conditions, including systemic inflammation, and inflammatory conditions affecting the large portions of or the whole body, includes reducing or downregulating the expression of cytokines and/or iNOS.

### HDAC and HDAC Inhibitors

In eukaryotic cells chromatin associates with histones to form nucleosomes. Each nucleosome consists of a protein octamer made up of two copies of each of histones H2A, H2B, H3 and H4. DNA winds around this protein core, with the basic amino acids of the histones interacting with the negatively charged phosphate groups of the DNA.

The most common posttranslational modification of these core histones is the reversible acetylation of the ε-amino groups of the conserved, highly basic N-terminal lysine residues. Reversible acetylation of histones is a regulator of gene expression. The acetylation state of histones determines whether the chromatin is in a condensed, transcriptionally silent state, or in a form more accessible to the transcription machinery of the cell. In general, hyperacetylation of histone proteins is associated with transcriptional activation of genes. Inhibition of HDACs results in the accumulation of hyperacetylated histones, which results in a variety of cellular responses.

Mammalian HDACs are divided into three major classes based on their structural or sequence homologies to the three distinct yeast HDACs: Rpd3 (class I), Hda1 (class II), and Sir2/Hst (class III). Class I and class II histone deacetylases (HDACs) are zinc containing hydrolase enzymes. The division of the proteins into classes I and II is based on protein size, sequence similarity, and organization of the protein domains.

Class I HDACs include: HDAC1 (GenBank Accession Number NP_{—}004955; Wolffe, A. P., Science 272, 5260, 371-372, 1996); HDAC2 (GenBank Accession Number NP_{—}001518; Furukawa, et al., Cytogenet. Cell Genet. 73; 1-2, 130-133, 1996); HDAC3 (GenBank Accession Number NP_{—}003874; Yang, et al., J. Biol. Chem. 272, 44, 28001-28007, 1997); HDAC8 (GenBank Accession Number NP_{—}060956; Buggy, et al., Biochem J. 350 Pt 1, 199-205, 2000); HDAC11 (GenBank Accession Number NP_{—}079103; Gao, L. et al., J. Biol. Chem. 277, 28, 25748-25755, 2002). The Hda1 homologous class II includes HDACs 4, 5, 6, 7, 9 (9a and 9b), and 10. The Sir2/Hst homologous class III includes SIRs T1, 2, 3, 4, 5, 6, and 7.

Recent studies revealed an additional family of cellular factors that possesses intrinsic HDAC activities. These appear to be non-histone proteins that participate in regulation of the cell cycle, DNA repair, and transcription. A number of transcription coactivators, including but not limited to, p400AF, BRCA2, and ATM-like proteins, function as HDACs. Some transcriptional repressors exhibit HDAC activities in the context of chromatin by recruiting a common chromatin-modifying complex. For instance, the Mas protein family (MasI, MxiI, Mad3, and Mad4) comprises a basic-helix-loop-helix-loop-helix-zipper class of transcriptional factors that heterodimerize with Max at their DNA binding sites. Mad:Max heterodimers act as transcriptional repressors at their DNA binding sites through recruitment of “repressor complexes.” Mutations that prevent interaction with either Max or the msin3 co-repressor complex fail to arrest cell growth. Accordingly, HDAC inhibitor used herein refers to any agent capable of inhibiting the HDAC activity from any of the proteins described above.

Inhibitors of HDAC have been studied for their therapeutic effects. For example, butyric acid and its derivatives, including sodium phenylbutyrate, have been reported to induce apoptosis in vitro in human colon carcinoma, leukemia and retinoblastoma cell lines.

### Pan-HDAC Inhibitors

By way of non-limiting example, pan-HDAC inhibitors (any of which are optionally used to treat the conditions and symptoms described herein) include short-chain fatty acids such as butyrate, 4-phenylbutyrate or valproic acid; hydroxamic acids such as suberoylanilide hydroxamic acid (SAHA), biaryl hydroxamate A-161906, bicyclic aryl-N-hydroxycarboxamides, CG-1521, PXD-101, sulfonamide hydroxamic acid, LAQ-824, oxamflatin, scriptaid, m-carboxy cinnamic acid bishydroxamic acid, trapoxin-hydroxamic acid analogue, trichostatin A, trichostatin C, m-carboxycinnamic acid bis-hydroxamideoxamflatin (CBHA), ABHA, Scriptaid, pyroxamide, and propenamides; epoxyketone-containing cyclic tetrapeptides such as trapoxins, apidicin, depsipeptide, HC-toxin, chlamydocin, diheteropeptin, WF-3161, Cyl-1 and Cyl-2; benzamides or non-epoxyketone-containing cyclic tetrapeptides such as FR901228, apicidin, cyclic-hydroxamic-acid-containing peptides (CHAPs), benzamides, MS-275 (MS-27-275), and CI-994; depudecin; PXD101; organosulfur compounds; and aroyl-pyrrolylhydroxy-amides (APHAs).

In some embodiments, the pan-HDAC inhibitor is Compound 1, SAHA (Zolinza), trichostatin A, MS-275, LBH-589, PXD-101, MGCD-0103, JNJ-26481585, R306465 (J&J), or sodium butyrate.

Compound 1 is a novel, orally dosed, hydroxamic acid-based HDAC inhibitor that inhibits all Class I and Class II HDAC isoforms with greatest potency against HDACs 1 and 3 (IC₅₀ 7-8 nM). Compound 1 has the following structure:

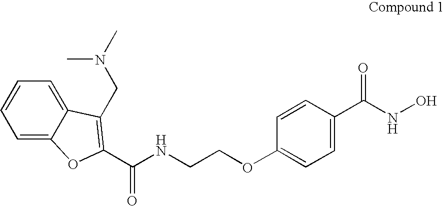

In some embodiments, the pan-HDAC inhibitor is a compound selected from a compound or formula disclosed in US publication no. 20070105939; US publication no. 20080139547; US publication no. 20070293540; US publication no. 20050187261; U.S. Pat. No. 7,368,572; U.S. Pat. No. 7,368,476; WO 2006/069096; WO 2005/097770; U.S. Pat. No. 7,276,612; U.S. Pat. No. 7,420,089; U.S. Pat. No. 7,482,466; U.S. Pat. No. 7,517,988; WO 04/092115; WO 05/019174; the disclosures of these references are herein incorporated in their entirety.

In some embodiments, the pan-HDAC inhibitor is a compound of Formula (I):

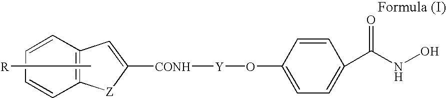

wherein:

### Z is S, O, or NH;

Y is alkylene optionally substituted with cycloalkyl, optionally substituted phenyl, alkylthio, alkylsulfinyl, alkylsulfonyl, optionally substituted phenylalkylthio, optionally substituted phenylalkylsulfonyl, hydroxyl, or optionally substituted phenoxy;
R is one or two optional substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenyl, optionally substituted phenoxy, optionally substituted phenylalkyloxy, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)ₙR^{a} (where n is 0, 1 or 2 and R^{a} is hydroxyalkyl or optionally substituted phenyl), -alkylene-NR^{e}-alkyleneCONR^{c}R^{d} (where R^{c} is hydroxyl and R^{d} and R^{e} are independently hydrogen or alkyl), or carboxyalkylaminoalkyl;
or a pharmaceutically acceptable salt thereof.

In some embodiments, the compound of Formula (I) is modified such that: Z is O (benzofuranyl), NH (indolyl), or S (benzothiofuranyl); and Y is —CH₂CH₂—.

In some embodiments, the benzofuranyl group of Formula (I) is monosubstituted. In some embodiments, the indolyl group of Formula (I) is monosubstituted. In some embodiments, the benzothiofuranyl group of Formula (I) is monosubstituted.

In some embodiments, the substituent on the benzofuranyl group, the indolyl group, or the benzothiofuranyl group is N,N-dimethylaminomethyl, N,N-diethylaminomethyl, 2-fluorophenoxymethyl, 3-fluorphwenoxymethyl, 4-fluorophenoxymethyl, hydroxyl-4-yloxymethyl, 2,4,6-trifluorophenoxy-methyl, 2-oxopyridin-1-ylmethyl, 2,2,2-trifluorethoxy-methyl, 4-imidazol-1-ylphenoxy-methyl, 4-[1.2.4]-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenyl)ethyl, N-methyl-N-benzylaminomethyl, N-methyl-N-2-phenylethylaminomethyl, 3-hydroxypropyl-thiomethyl, 3-hydroxypropylsulfinylmethyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylaminomethyl.

In some embodiments, the pan-HDAC is Compound 1. In some embodiments, the pan-HDAC is a pharmaceutically acceptable salt of Compound 1. In some embodiments, the pan-HDAC is the HCl of Compound 1.

Additional pharmaceutically acceptable salts of pan-HDAC inhibitor compounds include:

(a) salts formed when the acidic proton of the pan-HDAC inhibitor compound is replaced by a metal ion, such as for example, an alkali metal ion (e.g. lithium, sodium, potassium), an alkaline earth ion (e.g. magnesium, or calcium), or an aluminum ion, or is replaced by an ammonium cation (NH₄⁺);

(b) salts formed by reacting the pan-HDAC inhibitor compound with a pharmaceutically acceptable organic base, which includes alkylamines, such as ethanolamine, diethanolamine, triethanolamine, tromethamine, N-methylglucamine, dicyclohexylamine, tris(hydroxymethyl)methylamine, and salts with amino acids such as arginine, lysine, and the like;

(c) salts formed by reacting the pan-HDAC inhibitor compound with a pharmaceutically acceptable acid, which provides acid addition salts. Pharmaceutically acceptable acids include hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Additional pharmaceutically acceptable salts include those described in Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002.

The compounds presented herein include all diastereomeric, enantiomeric, and epimeric forms. For compounds described herein that exist as tautomers, all tautomers are included within the formulas described herein. Further, the compounds described herein may be formed as, and/or used as, pharmaceutically acceptable salts.

Exemplary synthetic methods useful for synthesizing these compounds include, for example, those disclosed in Nogrady (1985) Medicinal Chemistry A Biochemical Approach, Oxford University Press, New York, pages 388-392; Silverman (1992); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989).

### The Use of Pan-HDAC Inhibitors for Treating Cytokine-Modulated Health Conditions

In some embodiments, a subject with non-localized inflammatory conditions (or any symptoms associated with such inflammation), including systemic inflammation, and inflammatory conditions affecting the large portions of or the whole body, is treated with a therapeutically effective amount of a pharmaceutical composition comprising a compound of Formula (I) or pharmaceutically acceptable salts thereof.

In some embodiments, a therapeutically effective amount of a pharmaceutical composition comprising a compound of Formula (I) or pharmaceutically acceptable salts thereof reduces the secretion of pro-inflammatory cytokines including, but not limited to, IL-1β. In some embodiments, treatment with a therapeutically effective amount of a pharmaceutical composition comprising a compound of Formula (I) or pharmaceutically acceptable salts thereof can, in a dose dependent fashion, decrease lipopolysaccharide (LPS) and/or ATP stimulated secretion of IL-1β from purified human peripheral blood mononuclear cells (PBMCs). In some embodiments, a therapeutically effective amount of a pharmaceutical composition comprising a compound of Formula (I) or pharmaceutically acceptable salts thereof can decrease the secretion of IL-1β from the monocyte cell line THP-1. In some embodiments, the EC₅₀ for inhibition ranges from 0.5 μM to 5 μM.

In some embodiments, a therapeutically effective amount of a pharmaceutical composition comprising a compound of Formula (I) or pharmaceutically acceptable salts thereof is administered to a subject to decrease the systemic levels of one or more inflammatory cytokines including, e.g., IL-1β, IL-6, IL-18, TNF-α, MCP-1, or MIP-1α.

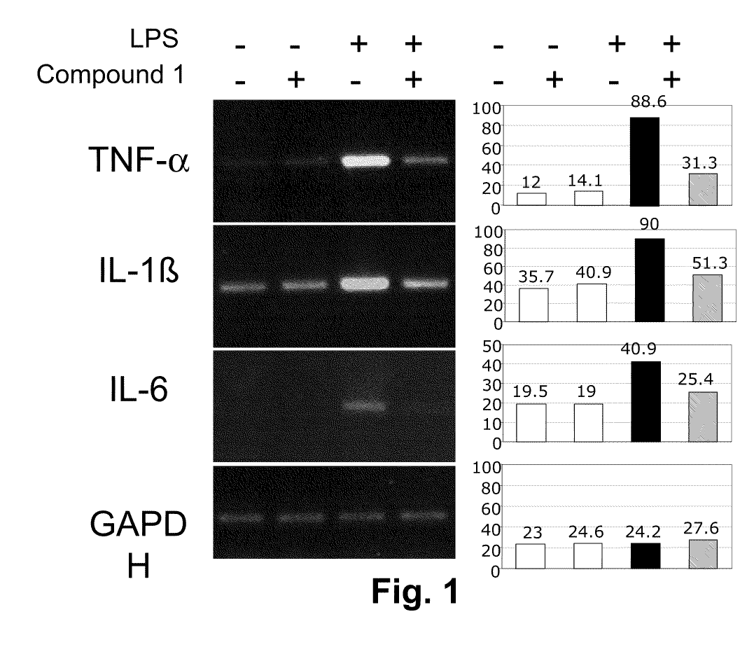

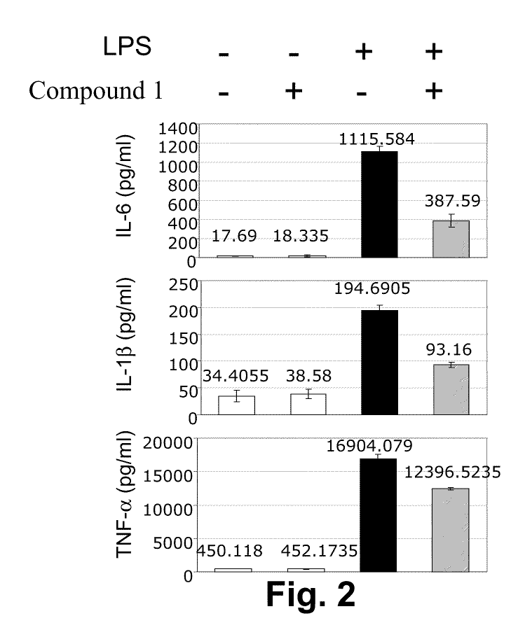

FIG. 1 and FIG. 2 provide evidence that treatment with the pan-HDAC inhibitor Compound 1 reduces the expression of TNF-α, IL-1β, and IL-6. Treatment with LPS induces an inflammatory response in cells. However, when cells treated with LPS were also treated with Compound 1 the expression of TNF-α, IL-1β, and IL-6 decreased.

The production and secretion of IL-1β is via a non-classical pathway of protein secretion involving potassium efflux, the autocatalytic processing of procaspase-1, the cleavage by active caspase-1 of the IL-1β precursor, the influx of calcium ions, and the activation of specific phospholipases including PLA-2. In some embodiments, pan-HDAC inhibitor compounds described herein inhibit one or more steps in this secretory pathway.

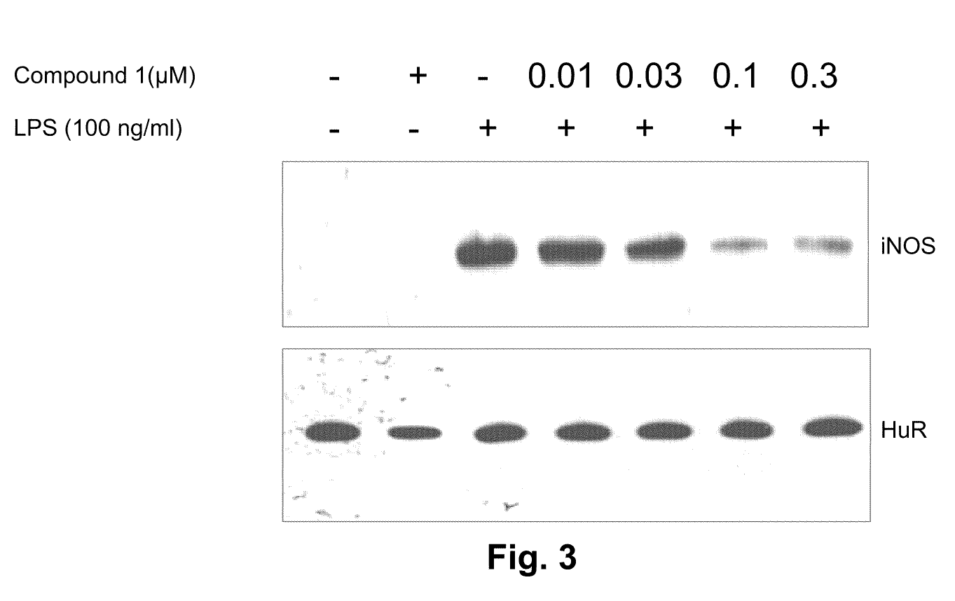

FIG. 3 provides evidence that treatment with the pan-HDAC inhibitor Compound 1 surprisingly and unexpectedly decreases blood pressure in a subject. It has been shown that a reduction in nitric oxide derived from inducible nitric oxide synthase (iNOS) decreases hypotension. (See, Selective iNOS inhibition prevents hypotension in septic rats while preserving endothelium-dependent vasodilation, Anesthesia and analgesia, 2001, vol. 92, no 3, pp. 681-687). Treatment with LPS causes an increase in iNOS expression. When cells are treated with both LPS and Compound 1 iNOS expression decreases in a dose-dependant manner. Thus, treatment with a therapeutically effective amount of a pharmaceutical composition comprising a compound of Formula (I) or pharmaceutically acceptable salts thereof, such as Compound 1, can not only decrease inflammation it can also decrease hypotension. A decrease in hypotension in a subject suffering from sepsis can prolong the subject's life allowing for further treatment of the sepsis.

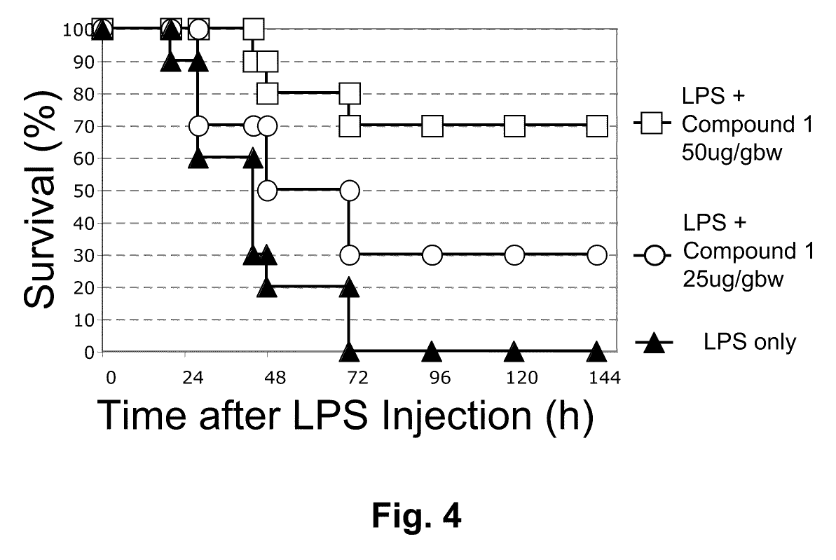

FIG. 4 provides evidence that a subject's overall chances of survival of sepsis increase with treatment by the pan-HDAC inhibitors described herein, for example Compound 1. At 72 hours after induction of sepsis by treatment with LPS, a subject has a 0% chance of survival. However, when the subject is also treated with 25 μg/gram of body weight of Compound 1 the subject's chances of survival at 72 hours are 30% and remain steady through 144 hours. Treatment with 50 μg/gram of body weight of Compound 1 increases the subject's chances of survival to 70%. This remains constant through 144 hours.

### Examples of Pharmaceutical Compositions and Methods of Administration

In some embodiments, a pharmaceutical composition comprising one or more pan-HDAC inhibitors described herein is administered to a subject having a non-localized inflammatory condition (or any symptoms associated with such inflammation), including systemic inflammation, and inflammatory conditions affecting the large portions of or the whole body. In some embodiments, the subject is a human patient. In some embodiments, the pharmaceutical compositions further comprise various excipients. Any pharmaceutically appropriate excipients may be used. For a list of excipients see Remington: The Science and Practice of Pharmacy, 19th Ed. (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, 7th Ed. (Lippincott Williams & Wilkins 1999).

The pharmaceutical compositions described herein may be formulated for administration to a subject via any conventional means including, but not limited to, oral, parenteral (e.g., intravenous, subcutaneous, or intramuscular), buccal, intranasal, rectal, intravaginal, or transdermal administration routes.

The therapeutically-effective amount of the pharmaceutical compositions described herein will vary depending upon factors such as, but not limited to, the particular compound, disease or condition and its severity, the identity (e.g., weight) of the subject in need of treatment, and the method of administration. In some embodiments, doses employed for adult human treatment will be in the range of about 0.02 to about 5000 mg per day. In some embodiments, the dose will range from about 1 to about 1500 mg per day. In some embodiments, the dose will range from about 10 to about 500 mg per day. In some embodiments, the desired dose is presented in a single dose or as divided doses administered simultaneously (or over a short period of time) or at appropriate intervals, for example as two, three, four or more sub-doses per day.

In some embodiments, the pharmaceutical compositions described herein are administered in combination with one or more additional therapeutic agents in order to treat a subject/patient having a non-localized inflammatory condition (or any symptoms associated with such inflammation), including systemic inflammation, and inflammatory conditions affecting the large portions of or the whole body. In some embodiments, is administered in combination with one or more of the following therapeutic agents: immunosuppressants (e.g., tacrolimus, cyclosporin, rapamicin, methotrexate, cyclophosphamide, azathioprine, mercaptopurine, mycophenolate, or FTY720), antibiotics (e.g. levofloxacin, amoxycillin), glucocorticoids (e.g., prednisone, cortisone acetate, prednisolone, methylprednisolone, dexamethasone, betamethasone, triamcinolone, beclometasone, fludrocortisone acetate, deoxycorticosterone acetate, aldosterone), non-steroidal anti-inflammatory drugs (e.g., salicylates, arylalkanoic acids, 2-arylpropionic acids, N-arylanthranilic acids, oxicams, coxibs, or sulphonanilides), Cox-2-specific inhibitors (e.g., valdecoxib, celecoxib, or rofecoxib), leflunomide, gold thioglucose, gold thiomalate, aurofin, sulfasalazine, hydroxychloroquinine, minocycline, TNF-α binding proteins (e.g., infliximab, etanercept, or adalimumab), abatacept, anakinra, interferon-β, interferon-γ, interleukin-2, allergy vaccines, antihistamines, antileukotrienes, beta-agonists, theophylline, or anticholinergics.

### EXAMPLES

### Example 1

### Compound 1 Decreases Expression of Cytokine mRNA

RAW cells were treated with 0.1 μM of Compound 1 for 30 min, then 100 ng/ml LPS was added for 6 hours. RT-PCR was performed (30 cycles), and PCR products were analyzed on a 1% agarose gel. Compound 1 decreased RNA levels of TNF, IL-1β, and IL-6, but not GAPDH. (FIG. 1)

### Example 2

### Compound 1 Decreases Levels of Secreted Cytokine Protein

RAW cells were treated as with 0.1 μM Compound 1 for 30 min and then LPS various amounts of 0.1 uM of Compound 1 for 30 min, LPS 100 ng/ml for 16 hr. Culture media was collected and analyzed by ELISA for TNFα, IL-1β and IL-6 proteins (n=3±S.D.). Compound 1 decreases cytokine expression. (FIG. 2).

### Example 3

### Compound 1 Decreases iNOS Expression

RAW cells were treated with various amounts of Compound 1 for 30 min, and LPS 100 ng/ml was added to the cells for 16 hours. Cell lysates were analyzed by Western blot with antibodies to the inducible nitric oxide synthase (iNOS) (above) and HuR (below) as a negative control. Compound 1 decreases iNOS expression. (FIG. 3).

### Example 4

### Compound 1 Decreases Mortality from LPS

RAW cells were treated as with 0.1 μM Compound 1 for 30 min and then LPS various amounts of 0.1 uM of Compound 1 for 30 min, LPS 100 ng/ml for 16 hr. Culture media was collected and analyzed by ELISA for TNFα, IL-1β and IL-6 proteins (n=3±S.D.). Compound 1 decreases cytokine expression. (FIG. 4).

### Example 5

### Treatment of a Patient with Sepsis

A postpartum female patient is presented with signs of sepsis. The body temperature of the patient is 100.7° F.; she has a respiratory rate of greater than 20 breaths per minute; and she has a white blood cells count cell count of 17,000/_L, with 40% neutrophils and 56% band cells. Infection with Streptococcus is confirmed by gram stain and culture. The patient is diagnosed with Group A streptococcal puerperal sepsis. The patient is placed on intravenous drip of Clindamycin plus a β-lactam antibiotic and is given Compound 1 in an oral dosage form. The patient's blood pressure is measured before treatment and the patient is classified as hypotensive. The patient's blood pressure should return to normal with the given therapy.

The examples and embodiments described herein are for illustrative purposes only and various modifications or changes suggested to persons skilled in the art are to be included within the spirit and purview of disclosure and scope of the appended claims.

What is claimed is:

1. A method for treating sepsis in a subject comprising administering to the subject a pharmaceutical composition comprising a therapeutically effective amount of a pan-HDAC inhibitor to the subject in need thereof.

2. The method of claim 1, wherein the pan-HDAC inhibitor is a short-chain fatty acid pan-HDAC inhibitor, hydroxamic acid pan-HDAC inhibitor, epoxyketone-containing cyclic tetrapeptide pan-HDAC inhibitor, benzamide pan-HDAC inhibitor, cyclic-hydroxamic-acid-containing peptide (CHAP) pan-HDAC inhibitor, benzamide pan-HDAC inhibitor, depudecin, organosulfur pan-HDAC inhibitor, or an aroyl-pyrrolylhydroxy-amide (APHA) pan-HDAC inhibitor.

3. The method of claim 2, wherein the pan-HDAC inhibitor is butyrate, 4-phenylbutyrate, valproic acid, suberoylanilide hydroxamic acid (SAHA), biaryl hydroxamate A-161906, bicyclic aryl-N-hydroxycarboxamides, CG-1521, PXD-101, sulfonamide hydroxamic acid, LAQ-824, oxamflatin, scriptaid, m-carboxy cinnamic acid bishydroxamic acid, trapoxin-hydroxamic acid analogue, trichostatin A, trichostatin C, m-carboxycinnamic acid bis-hydroxamideoxamflatin (CBHA), azelaic bishydroxamic acid (ABHA), Scriptaid, Sirtinol, pyroxamide, trapoxins, apidicin, depsipeptide, HC-toxin, chlamydocin, diheteropeptin, WF-3161, Cyl-1 and Cyl-2, FR901228, apicidin, cyclic-hydroxamic-acid-containing peptide (CHAP), MS-275 (MS-27-275), CI-994, depudecin, PXD101, an aroyl-pyrrolylhydroxy-amide (APHA), LBH-589, MGCD-0103, JNJ-26481585, R306465 (J&J), or sodium butyrate.

4. The method of claim 2, wherein the pan-HDAC inhibitor is a compound with the structure of Formula (I): 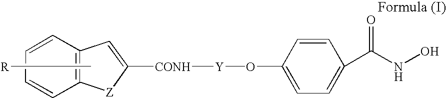
wherein
Z is S, O, or NH;
Y is an alkylene optionally substituted with cycloalkyl, optionally substituted phenyl, alkylthio, alkylsulfinyl, alkylsulfonyl, optionally substituted phenylalkylthio, optionally substituted phenylalkylsulfonyl, hydroxyl, or optionally substituted phenoxy;
R is one or two optional substituents independently selected from alkyl, halo, haloalkyl, alkoxy, alkoxyalkyl, hydroxyalkoxy, hydroxyalkoxyalkyl, alkoxyalkyloxy, alkoxyalkyloxyalkyl, aminoalkyl, aminoalkoxy, haloalkoxy, haloalkoxyalkyl, optionally substituted phenyl, optionally substituted phenoxy, optionally substituted phenylalkyloxy, optionally substituted phenylalkyl, optionally substituted phenyloxyalkyl, optionally substituted heteroaryl, optionally substituted heteroaralkyloxy, optionally substituted heteroaryloxyalkyl, optionally substituted heterocycloalkylalkyl, optionally substituted heterocycloalkyloxy, optionally substituted heterocycloalkylalkyloxy, -alkylene-S(O)ₙR^{a} (where n is 0, 1 or 2 and R^{a} is hydroxyalkyl or optionally substituted phenyl), -alkylene-NR^{e}-alkyleneCONR^{c}R^{d} (where R^{c} is hydroxyl and R^{d} and R^{e} are independently hydrogen or alkyl), or carboxyalkylaminoalkyl, or a pharmaceutically acceptable salt thereof.

5. The method of claim 4, wherein Z is O, NH, or S; and Y is —CH₂CH₂—.

6. The method of claim 5, wherein the benzofuranyl group is monosubstituted.

7. The method of claim 5, wherein the indolyl group is monosubstituted.

8. The method of claim 5, wherein the benzothiofuranyl group is monosubstituted.

9. The method of claim 6, wherein the monosubstitution is with a group selected from N,N-dimethylaminomethyl, N,N-diethylaminomethyl, 2-fluorophenoxymethyl, 3-fluorphwenoxymethyl, 4-fluorophenoxymethyl, hydroxyl-4-yloxymethyl, 2,4,6-trifluorophenoxy-methyl, 2-oxopyridin-1-ylmethyl, 2,2,2-trifluorethoxy-methyl, 4-imidazol-1-ylphenoxy-methyl, 4-[1.2.4]-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenyl)ethyl, N-methyl-N-benzylaminomethyl, N-methyl-N-2-phenylethylaminomethyl, 3-hydroxypropyl-thiomethyl, 3-hydroxypropylsulfinylmethyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylaminomethyl.

10. The method of claim 2, wherein the pan-HDAC inhibitor is Compound 1: 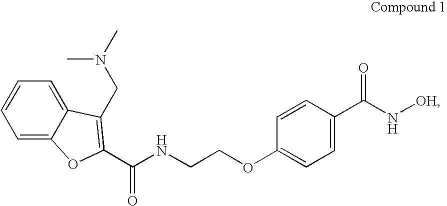
or a pharmaceutically acceptable salt thereof.

11. The method of claim 10, wherein the pan-HDAC inhibitor is the HCl salt of Compound 1.

12. The method of claim 1 in which the expression of iNOS decreases or is down-regulated following administration of the pharmaceutical composition.

13. The method of claim 1 in which the concentration of nitric oxide in the blood of the subject decreases following administration of the pharmaceutical composition.

14. The method of claim 1 in which after administration of the pharmaceutical composition, the blood pressure of the subject increases.

15. The method of claim 1 in which following administration of the pharmaceutical composition the expression of one or more cytokines has decreased or is down-regulated.

16. The method of claim 14, wherein the one or more cytokines is selected from the group consisting of: IL-1, IL-6, TNF-α, any isoforms thereof, and any combinations thereof.

17. The method of claim 1, wherein the pharmaceutical composition is administered in combination with one or more additional therapeutic agents.

18. The method of claim 17, wherein the one or more additional therapeutic agents is selected from the group consisting of: immunosuppressants, antibiotics, glucocorticoids, non-steroidal anti-inflammatory drugs, Cox-2-specific inhibitors, disease modifying antirheutetic drugs, TNF-α binding proteins, beta-agonists, and any combinations thereof.

19. The method of claim 1, wherein the subject is a human.
